(19) United States
(12) Patent Application Publication
(10) Pub. No.: US 2004/0037736 A1
(43) Pub. Date: Feb. 26, 2004

(54) Plasma sterilisation system

(76) Inventors: Francois Perruchot, Issy Les Moulineaux (FR); Marie-Pierre Jaffrezic, Velizy (FR); Philippe Destrez, Meudon (FR); Severine Bousquet, Gif Sur Yvette (FR)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/380,088

(22) PCT Filed: Sep. 13, 2001
(86) PCT No.: PCT/FR01/02842

(30) Foreign Application Priority Data
Sep. 15, 2000 (FR) 00/11829

(51) Int. Cl. A61L 2/00; A61L 9/00; A61L 11/00

(52) U.S. Cl. 422/22; 422/1; 422/4; 422/121; 422/292; 422/305; 422/306; 422/907

(57) ABSTRACT

A method of sterilizing at least one article by means of a plasma and in the presence of humidity using a non-biocidal gas containing oxygen and nitrogen, the article being placed outside the discharge in a sealed treatment enclosure that is subjected substantially to atmospheric pressure, the method comprising the following steps: introducing the humidified non-biocidal gas into the treatment enclosure; creating a first plasma discharge A for a determined duration enabling the effectiveness of the sterilizing species created during the following stage to be guaranteed within the entire enclosure; creating a second plasma discharge B during a determined duration enabling said article to be sterilized; and rinsing the treatment enclosure during a determined duration so as to guarantee that it contains a non-polluting atmosphere when the enclosure is subsequently opened. Preferably, discharge of the first plasma and humidity introduction take place simultaneously and the first and second plasma discharges can overlap so that creation of the second plasma begins before creation of the first plasma terminates. The present invention also provides various devices for implementing the method and serving in particular to sterilize all types of medical article.

The present invention relates to the general field of sterilizing articles and surfaces of any kind and of any shape, and it relates more particularly to a method and to various devices for plasma sterilization operating at ambient temperature and at atmospheric pressure.

### PRIOR ART

Sterilization corresponds to a well-defined level of quality in medical and food industry circles. In medical circles, it means that all microorganisms of any kind whatsoever are destroyed. According to the European Pharmacopeia, an article can be considered as being sterile if the probability of a viable microorganism being present thereon is less than or equal to 10⁻⁶. Sterilization time is the time needed to sterilize a “normally contaminated” article, i.e. containing 10⁶ spores of bacteria. Thus, sterilizing an article corresponds to reducing an initial population of bacterial spores present on said article from 10⁶ spores to 10⁻⁶ spores, giving a logarithmic reduction of 12 decades. The time needed to achieve reduction by 1 decade is by definition referred to as the decimal reduction time, written D. It is a fundamental variable for characterizing a sterilization method.

At present, numerous methods exist that enable articles to be made and kept sterile. The article by Philip M. Schneider published in Vol. 77 of Tappi Journal in January 1994 at pages 115 to 119 gives a relatively exhaustive summary. Nevertheless, Mr. Schneider terminates his article with the observation that there does not exist at present any ideal method for sterilization at low temperature (less than 80° C.), i.e. a method which is highly effective, which acts quickly, and which presents a high degree of penetration, while also being non-toxic, and compatible with numerous materials, in particular organic materials, and that is capable of being implemented simply and at low cost.

In addition, the sterile state of an article must be maintained by specific packaging which must be compatible with the sterilization method used (permeable to the sterilizing agent) and must prevent microorganisms penetrating during transport and storage, in order to guarantee that an instrument is sterile when next used.

Present sterilization methods are based essentially on the effect of heat or on the action of biocidal gas.

An autoclave which relies on the action of humid heat at high temperature (at least 121° C.) is the method that is the most effective and the least expensive to implement, however it is unsuitable for sterilizing temperature-sensitive devices which are sensitive to heat and which are becoming more and more widespread, particularly in the medical field.

Sterilization methods using gases (ethylene oxide, formaldehyde, hydrogen peroxide) make use of the biocidal nature of a gas placed in a sterilization enclosure, and enable temperature-sensitive devices to be sterilized at low temperature. However, such methods present numerous drawbacks: the toxic nature of the gases in question requires complex utilization and inspection procedures; in some cases (e.g. when using plastics materials), it is essential to implement a stage during which the toxic gas is desorbed after sterilization has been performed; finally, the duration of the treatment often extends for several hours. Furthermore, it should be observed that the destructive effect is limited on certain kinds of bacterial spore (such as the spores of Bacillus stearothermopyhilus).

Thus, one known improvement to such methods of sterilization by means of biocidal gases consists in performing treatment at low pressure (a few torrs), thus encouraging the diffusion of the gas or the vaporization of an additional biocidal liquid throughout the sterilization enclosure. Similarly, sterilization methods can be optimized for low pressure by establishing cycles made up in an alternation of phases during which pressure is reduced or increased, and phases of plasma treatment, as disclosed in particular in French patent application No. FR 2 759 590 filed by the supplier SA Microondes Energie Systèmes.

Sterilization methods are also known that make use of a low pressure plasma and that can possibly serve to combine the sterilizing effects of the low pressure biocidal gas with the formation of reactive species (creation of O^{•} and OH^{•} radicals of ionized and/or excited species) from a mixture of biocidal gas such as H₂O₂ or a mixture of non-biocidal gas (generally merely O₂, H₂, H₂O, N₂, or a rare gas such as argon). In most cases, low pressure plasmas involve microwave or radiofrequency plasmas.

Low pressure plasma sterilization can be very effective in the space where the plasma is created (the gap between the electrodes), however apart from the fact that the sterilization zone is then very small (only a few centimeters (cm) in height), the characteristics of the plasma depend very strongly on the dielectric constant, the nature, and the size of the article to be sterilized. Under such circumstances, the plasma prevents genuinely uniform treatment being applied to the entire surface, and it also has highly corrosive effects on the articles that are to be sterilized.

To improve such a method, it is necessary to separate the plasma production zone from the treatment zone (sterilization is then said to be “post-discharge” sterilization) in order to avoid too great an interaction between the plasma and the article to be sterilized. Such separation of the plasma production zone from the sterilization zone is easy to achieve in a low pressure method since the low pressure limits the extent to which unstable species can recombine, so the lifetimes of the radicals produced by the plasma at such pressures (about 1 torr) are long, thus enabling them to reach the articles to be sterilized.

The drawbacks of low pressure plasma sterilization methods are nevertheless still numerous, and in the absolute fairly similar to those which exist when performing sterilization by means of gas only: the complete system comprising both an enclosure that withstands a vacuum and also a plasma generator is expensive; the devices implementing such methods are complex, thereby limiting possible applications; treatment time is increased due to the procedures associated with low pressure treatment (the time needed to evacuate the enclosure, which enclosure is often of large volume, and the time needed subsequently to return to atmospheric pressure serves to increase total treatment time); it is not possible to sterilize wet articles; and the method is incompatible with certain materials.

Thus another known method using the post-discharge principle consists in performing treatment with ozone at atmospheric pressure using an appropriate device referred to as an “ozoner”. The treatment is similar to post-discharge plasma sterilization at atmospheric pressure with a vector gas that does not contain any humidity so as to encourage the creation of oxygen-containing ozone. Nevertheless, the biocidal action of ozone, which is used above all for the purpose of disinfecting water and waste gas, is rather limited in terms of sterilization power. To obtain better performance, it is generally necessary for the ozone (03) to be associated with a disinfectant agent (e.g. ClO₃ in order to form ClO₃ that presents bactericidal action when in the gaseous phase). It is also possible to humidify the ozone-containing gas leaving the ozoner or merely to moisten the articles to be sterilized in order to facilitate the biocidal action of the ozone-containing gas, as illustrated by U.S. Pat. No. 5,120,512 (Masuda). In general, for plasma methods based on simple, non-biocidal gases, separation between the plasma production zone and the treatment zone limits the effectiveness of the method, since only those species that are of medium to long lifetime are still active in the vicinity of the article. Unfortunately, these are the species that are less reactive than those that present a short lifetime, so it is necessary to increase their concentration and the duration of treatment. For example, it is known that nearly 3 hours of treatment are needed to sterilize spores of Bacillus subtilis in the presence of moisture using ozone at a concentration of 1500 parts per million (ppm). The ozone concentrations used in sterilizers based on ozoners are much higher, lying typically in the range 10,000 ppm to 80,000 ppm. Production at that level requires the use of devices that are complex, and a high concentration of ozone increases the irreversible damage to the surfaces and the materials of articles that are to be sterilized. In addition, the production of ozone at high concentration is subject to regulation and requires the use of a particularly efficient ozone destruction device at the outlet from the system, e.g. a device of the type that uses a catalyst or heat treatment.

In particular, in international patent application No. PCT/FR00/00644 filed in the name of the Applicant, the inventors propose a novel method of sterilization at atmospheric pressure and at ambient temperature using plasma in post-discharge. That method operates using a mixture of non-biocidal gases containing nitrogen and oxygen (e.g. air), with sterilization taking place in the presence of moisture at a relative humidity of more than 50%, and it makes it possible to avoid using complex vacuum-generating devices and to avoid relying on biocidal gases. Its simplicity enables it to be used for a variety of configurations enabling sterilization to be fragmented. It operates on the principle of a cycle made up of three successive stages as shown in FIG. 6. The first stage Ph1 corresponds to introducing a non-biocidal gas mixture containing a high percentage of humidity into the treatment enclosure. The second stage Ph2 starts when the level of humidity inside the enclosure is sufficient and it corresponds to a sterilization stage proper based on a plasma discharge creating species that have sporicidal action. The duration of this stage is determined by the level of decontamination that is desired. The last stage Ph3 corresponds to rinsing the enclosure and it marks the end of the treatment cycle.

The effectiveness of that method operating in post-discharge relies on the possibility of active species created by the plasma source being propagated all the way to the surface that is to be sterilized. Propagating species from one point to another within the enclosure involves both the actual propagation time and the surfaces that are encountered between the two points. Nevertheless, although such a method is generally satisfactory, in certain particular conditions of use, and in particular for treatment in enclosures of large volume or for the treatment of articles that are elongate in shape, it can be necessary either to distribute a number of sources along the sterilization zone so as to reduce the maximum distance from a source for each point on the surface, or else to provide within the enclosure suitable propagation zones enabling sterilizing species to be propagated. Both of those two solutions suffer from the consequence of increasing the cost of the treatment enclosure quite considerably and, in particular, of requiring a high voltage connection to be established between the various plasma sources, or of requiring the enclosure to be complex in shape.

### OBJECT AND DEFINITION OF THE INVENTION

The object of the present invention is thus to propose an improved method of sterilization, making it possible to optimize sterilization time as well as possible for all configurations, including for enclosures of large volume or articles that are elongate, and to do so without increasing the cost of the enclosure. Another object of the invention is to propose an improved method enabling the number of plasma sources needed for sterilization purposes to be decreased without making the manufacture of the enclosure more complex. Another object of the invention is to provide a method presenting a rate of spore destruction that is reasonable at low temperature. Yet another object is to provide a method that is not polluting, avoiding any need to handle dangerous substances, unlike the simplest known methods for sterilization at low temperature.

The invention provides a method of sterilizing at least one article by means of a plasma and in the presence of humidity using a non-biocidal gas containing oxygen and nitrogen, the article being placed outside the discharge in a sealed treatment enclosure that is subjected substantially to atmospheric pressure, the method being characterized in that it comprises the following steps:

introducing the humidified non-biocidal gas into the treatment enclosure;

creating a first plasma discharge A for a determined duration enabling the effectiveness of the sterilizing species created during the following stage to be guaranteed within the entire enclosure;

creating a second plasma discharge B during a determined duration enabling said article to be sterilized; and

rinsing the treatment enclosure during a determined duration so as to guarantee that it contains a non-polluting atmosphere when the enclosure is subsequently opened.

Thus, with the invention it is possible with a limited number of plasma sources to treat articles of elongate shape, or more generally to use enclosures of large volume. Said determined durations are also calculated as a function of the volume of the treatment enclosure and of the articles to be treated.

Advantageously, the end of the rinsing step is detected by a parameter crossing a minimum threshold. Advantageously, this parameter is relative humidity, and the concentration of ozone as measured by a multiparameter sensor placed at the outlet from the treatment enclosure.

In a preferred embodiment, the discharge of plasma A and the introduction of humidity are simultaneous, and the discharges of the first and second plasmas overlap in such a manner that the second plasma B begins to be created before the first plasma A ceases to be created. The discharges of the first and second plasmas may make use of the same plasma source. The first and second plasma discharges are preferably of different kinds so as to enable each of the stages to be optimized separately.

Advantageously, the flow rate of the non-biocidal gas differs between the various stages.

Advantageously, the choice of discharge conditions for the first and second plasmas is determined by the individual pattern of the voltage signal (alternating current (AC), damped AC, or direct current (DC)), the repetition frequency of the pattern, and the total reference current. Advantageously, the conditions used are controlled by detecting peak currents. This detection is preferably performed using a pass bandwidth of the same order as the frequency between pulses.

Advantageously, the pattern repetition frequency or a latency time between individual patterns is used to limit temperature rise in the reactor while conserving the same discharge conditions.

In a variant implementation, the increase in the temperature of the article is compensated by temperature-stabilization of an evaporation humidifier at a temperature which is slightly below the temperature of the article. In another variant implementation, such compensation is obtained by controlling the effectiveness of a vaporizer in such a manner as to maintain constant humidity at the article.

Advantageously, the high voltage supply is taken from a pulsed low voltage supply feeding a transformer that is used both as a filter and to step-up voltage. Advantageously, controlling the repetition rate of the low voltage pulses enables latency time to be adjusted. In a first method of use, the repetition frequency of the low voltage pulses is lower than the resonant frequency of the transformer. In a second method of use, the repetition frequency of the pulses is equal to the resonant frequency of the transformer.

In a first embodiment, power supply is regulated on the basis of a measurement of current, preferably DC for a DC voltage supply, and synchronous for an AC voltage supply, the current being measured by passing through resistance. The current may also be measured indirectly by measuring the charge on a capacitor. With a DC voltage supply, capacitor discharge is implemented by periodic grounding.

In another implementation, the power supply is regulated on the basis of measuring peak current. Advantageously, the signal used for regulation purposes is smoothed with a time constant longer than 100 milliseconds (ms), and preferably longer than 1 second (s).

The electrodes are made from a blade having one or more points placed parallel to a plane surface or a cylindrical surface that acts as a backing electrode.

In a preferred implementation, the number of points is selected to facilitate the use of the different discharge conditions that are desired during treatment.

In a preferred configuration envisaged for the device, it comprises a plurality of treatment enclosures, each treatment enclosure having at least one plasma production zone connected in optionally fixed manner to at least one sterilization zone, the plasma production zones being connected to a common central unit containing at least the first source of non-biocidal gas, the humidification chamber, the system for recovering gas residues, and as many high voltage power supplies as there are outlets enabling enclosures to be treated simultaneously with different discharge conditions being applied thereto.

In an embodiment, the sterilization zone is pressurized to a small extent so as to enable flow to take place in fine capillaries.

In an embodiment, there is a multiparameter sensor for each connection path enabling the composition of the gas leaving an enclosure to be monitored prior to filtering.

### BRIEF DESCRIPTION OF THE DRAWINGS

Other characteristics and advantages of the present invention appear better from the following description given by way of non-limiting indication and made with reference to the accompanying drawings, in which:

FIGS. 1A, 1B, and 1C are timing diagrams showing the improved plasma sterilization method of the invention;

FIG. 2 is a block diagram of a plasma sterilization device of the invention;

FIG. 3 is a block diagram of a high voltage power supply suitable for the FIG. 2 device;

FIG. 4 shows an embodiment of the FIG. 2 plasma sterilization device;

FIG. 5 shows an embodiment of a treatment enclosure in accordance with FIG. 2;

FIG. 5A is an exploded view of FIG. 5 showing a particular configuration of the discharge zone; and

FIG. 6 is a timing diagram showing the various steps in a prior art plasma sterilization method.

### DESCRIPTION OF A PREFERRED EMBODIMENT

The invention relates to an improved sterilization method having sporicidal effectiveness tested in particular on the bacterial spores that are considered by the European Pharmacopeia as being the most resistant: Bacillus subtilis and Bacillus stearothermopyhilus.

In general, the method uses a mixture of gases containing oxygen and nitrogen, from which a low temperature plasma is created having chemical species with sterilizing action on the article to be treated in the presence of humidity. The article to be treated is placed outside the space where discharge occurs, and treatment is performed at atmospheric pressure.

The plasma is a gas that has been partially activated by an electromagnetic source of sufficient energy. The species created in the plasma are ionized species (molecules or atoms), neutral species (such as radicals), or excited species. These gaseous species have increased reactivity which enables them to interact with the surfaces of the article(s) to be sterilized, thereby destroying microorganisms present on said surfaces. At atmospheric pressure for a plasma created from simple, non-biocidal gas, the most reactive species are those that have a short lifetime, so this effectiveness depends strongly on the distance between the zone in which the plasma is created and the article. The invention proposes modifying the treatment cycle as described in above-specified application PCT/FR00/00644 so that during treatment of an article of elongate shape, or more generally in an enclosure of large volume, effectiveness is guaranteed throughout the volume of the enclosure while using a minimum number of plasma sources. It is proposed to adapt the implementation of this new treatment cycle in particular to designing a specific high voltage power supply.

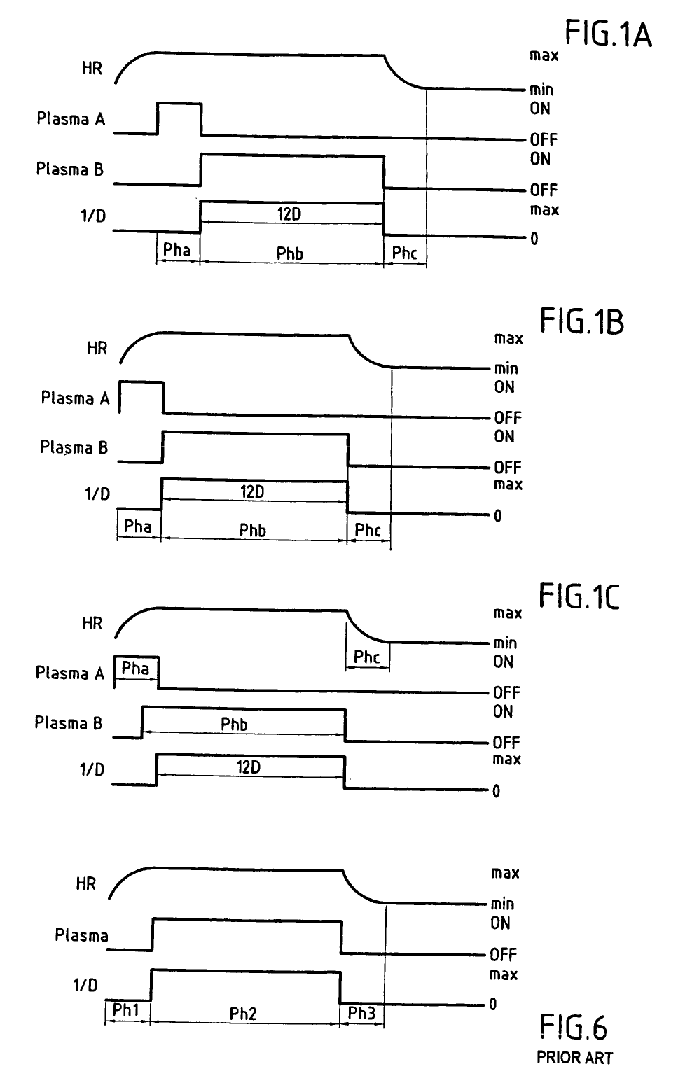

A preferred example of the treatment cycle in accordance with the invention is shown in FIGS. 1A to 1C. The cycle still comprises three successive stages, however these stages are now organized in different manner: a first stage Pha during which the enclosure is brought into equilibrium comprises discharging a first plasma A; a second stage Phb of actual treatment comprises discharging a second plasma B; and a third and last stage Phc comprises rinsing. Total cycle time is reduced by optimizing each of these three stages, taking account in particular of the effect of each stage on the following stage, if any.

The purpose of the first stage Pha is to bring the entire enclosure into conditions that enable the species to be created during the following stage Phb to propagate over all of the surfaces to be treated and to have sporicidal action within a reasonable length of time. As in the above-mentioned method, this stage necessarily includes introducing humidity in uniform manner, which is a minimum necessary condition for creating species during the second stage Phb. However, it further includes an optionally simultaneous discharge of a first plasma that does not necessarily have any significant sporicidal action but that is essential for bringing the enclosure into equilibrium prior to undertaking the following stage. Starting the first plasma while simultaneously introducing humidity also makes it possible to reduce significantly the total time required for treatment as can be seen in FIG. 1B. The duration of this stage is determined as a function of the volume of the enclosure and of the articles to be sterilized.

As shown in Table 1 below, following tests performed by the inventors, using the method in the above-mentioned international patent application, when sterilizing articles of very elongate shape such as endoscope channels, or articles of very large volume, a determined length of time TO elapses before the plasma begins to have any sporicidal effect. This time TO does not depend solely on a resistance time associated with the spores, but also on the nature of the walls of the enclosure.

TABLE 1
Stabilization time TO prior to sterilization (excluding humidity)
in a tube having an inside diameter of 25 millimeters (mm)

Equilibrium time [min]

| Distance [cm] | Glass | Stainless steel |
|---|---|---|
| 13 | 0 | 0 |
| 23 | 3 | 16 |
| 30 | 15 | 42 |

There thus exists a critical distance between the source and the surface to be treated beyond which this phenomenon of no immediate sporicidal effect appears. This stabilization time TO which needs to be taken into account in order to guarantee a sterile state at the end of the cycle therefore increases the total time needed for treatment.

The second stage Phb is the stage in which effective treatment takes place, and when the treatment is sterilization its duration is 12 D. D is measured on the basis of tests performed under the most unfavorable conditions on microorganisms that are considered as being the most resistant. The second plasma is optimized to maximize its sporicidal action while limiting damage to materials. When the gas production means make this possible, the second stage Phb can be advanced, i.e. it can begin before the end of the first stage Pha, as shown in FIG. 1C, thereby guaranteeing proper filling with plasma B before plasma A is stopped. In this configuration, the second stage Phb presents a duration that is longer than 12 D.

The last stage is to enable to the enclosure to return to an atmosphere that is compatible with storing the enclosure and with opening it at a later date, and this is done by causing a dry gas to circulate.

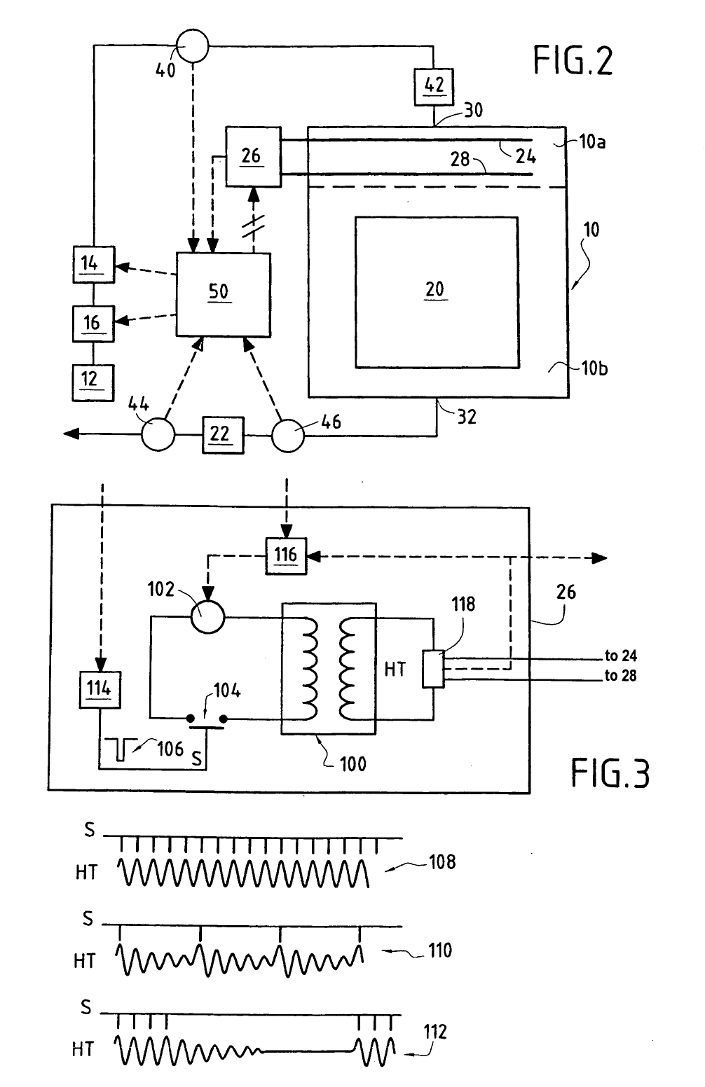

A block diagram of a plasma sterilization device implementing the improved method of the invention is shown in FIG. 2. The device is organized around a treatment enclosure 10 which is subdivided into two zones: a plasma production zone 20a within which a plasma is created by discharge between two electrodes; and a sterilization zone 10b in which the article to be treated is placed. The discharge is produced from a non-biocidal gas mixture provided by a gas source 12 via a humidifying chamber 14. The humidifying chamber possesses at least two ports, a maximum humidity port and a minimum humidity port referred to as a “dry” port. Selection between the two ports is performed as a function of which stage is in progress: a humid port during stage Phb and at least a portion of stage Pha, and a dry port during stage Phc. This treatment enclosure is completely closed.

The gas mixture contains hydrogen and nitrogen and its composition may vary as a function of the nature of the article to be sterilized. The aggressivity of the mixture relative to the materials constituting the articles that are to be sterilized depends on the oxygen content of the mixture. The gas mixture must contain at least 10% oxygen and 10% nitrogen in order to achieve an acceptable sporicidal effect, as shown by the table below (the samples contain spores of Bacillus subtilis):

TABLE 2
Comparing the effectiveness of discharge using a vector gas
containing argon or nitrogen, with measurement of the associated
ozone content (where R means that the sample is at the reference level)

| | 20% O₂ in N₂ | | | 20% O₂ in Ar | |
|---|---|---|---|---|---|
| RH = 80% | Living | | | Living | |
| Time [min] | spores | | O₃ [ppm] | spores | O₃ [ppm] |
| 0 | 16⁶ | (R) | 750 | 10⁶ (R) | 1000 |
| 10 | 10³ | | | 10⁶ (R) | |
| 20 | <10 | | | 10⁶ (R) | |

Advantageously the mixture is ambient air or air obtained from a compressor. The relative humidity (RH) in the sterilization zone during the second stage Phb, and thus in the vicinity of the articles to be treated, lies in the range 50% to 100%, and is advantageously greater than or equal to 70%. Table 3 below shows the importance of this parameter.

TABLE 3
Comparison of effectiveness for identical time
with RH lying in the range 20% to 80%
20 minute treatment

| Relative humidity [%] | Living spores | |
|---|---|---|
| 10 | 10⁶ | (R) |
| 20 | 10⁶ | (R) |
| 30 | 10⁶ | (R) |
| 40 | 10⁵ | |
| 50 | 10⁴ | |
| 60 | 10² | |
| 70 | <10 | |

The rate at which the gas mixture is admitted into the treatment enclosure 10 is adjusted as a function of the size and quantity of articles to be sterilized and as a function of the current stage, by means of a control device (e.g. a valve 16) placed at the outlet from the gas source 12 and serving to control its flow speed and its concentration.

The article to be sterilized 20 is placed on a support which must allow the sterilizing agent to flow over its entire surface, within the sterilization zone 10b, i.e. outside the zone where the plasma is produced (the interelectrode zone where the discharge is created).

In the example shown, a humid gas mixture is supplied and the plasma production zone includes both an inlet orifice for admitting the vector gas and two electrodes, namely a high voltage electrode 24 powered by a low frequency high voltage generator 26 and a ground electrode 28 which are designed to produce a “corona” electric discharge between them. A corona discharge is characterized by using two electrodes having radii of curvature that are very different. The increase in the electric filed close to the electrode having a small radius of curvature makes it possible to reduce the voltage needed for causing a discharge to appear while still making use of a voltage supply that can operate at low frequency since it does not make use of any resonance effect at the electrodes.

The vector gas admission orifice 30 is preferably situated close to the electrodes 24 and 28 so as to optimize the passage of the vector gas through the intereletrode space, with plasma production being located in said interelectrode space. A gas outlet orifice 32 is situated in the sterilization zone 10b downstream from the article to be treated in the natural flow direction of the gas.

A humidity and temperature sensor 40 is placed at the outlet of the humidifier in order to verify the quantity of water vapor present in the gas admitted to the discharge zone 10a. A bactericidal filter 42 is placed between the sensor and the inlet 30 in order to guarantee that the injected gas is sterile, particularly during the last stage Phc of rinsing.

The gaseous residue (effluent) that results from the discharge is exhausted via the outlet 32 to a recovery system 22 so as to avoid exceeding the limiting concentrations as set by regulations. For ozone, for example, the mean acceptable value of exposure on a workplace over a period of 8 hours is 0.1 ppm, according to the standard set by the Occupational Safety Health Administration. The gas leaving the recovery system 22 is analyzed, e.g. by means of an ozone sensor 44 in order to verify that filtering has operated properly prior to being exhausted to the outside.

A multiparameter sensor 46 is placed ahead of the filter 42 in order to analyze the gas leaving the reactor 10. This sensor is preferably sensitive to temperature, to humidity, and to the chemical composition of the gas, e.g. in terms of ozone content.

The measurements coming from the sensors 40, 44, and 46 and from the source 26 are centralized via a control member 50 which governs changeover between the various stages of the cycle by changing the reference values applied to the high voltage source 26, the valve 16, and the humidity 14.

The first and second stages during which plasma is created have different functions: the first plasma is used for bringing the system into equilibrium by creating chemical conditions that are favorable for sterilization, whereas the second plasma has a sporicidal effect, so it is preferable to have different types of discharge conditions available corresponding to these different chemical productions in order to be able to optimize the method. To obtain these different conditions, it is possible to use different plasma sources. The general purpose of the various solutions proposed below is to increase the number of discharge conditions that can be achieved using a given configuration so as to enable the method and the means used for controlling it to be optimized as much as possible. For example, for a given type of high voltage signal defined by its polarity, its waveform, and its frequency, the signal associated with data electrodes, the discharge conditions, and the production of chemical species depend on the intensity and the waveform of the current. The total current measured at the ground electrode contains various components associated with different physical effects. For example, for a positive DC voltage associated with a point-and-plane type of shape, the current below a voltage threshold is made up solely of a DC component associated to so-called “glow” conditions. At higher voltage, there is added pulsed type current made up of high amplitude pulses, typically of several milliamps (mA) and of short duration, typically 100 nanoseconds (ns), associated with “streamer” conditions. A higher voltage causes a DC component due to electric arcing to appear which corresponds to the appearance of a conductive channel through the gas.

The use of an AC voltage makes it possible to place a dielectric on one or other of the electrodes, preferably on the plane electrode, thus retarding the onset of electric arcing. This is referred to as dielectric barrier discharge (DBD) conditions. Intermediate energy conditions also occur, making it possible to pass through different chemical production zones. New pulses then appear at very high amplitude, typically several hundreds of milliamps (mA) and of short duration, typically 100 ms. This type of DBD discharge thus provides a high degree of flexibility in selecting the particular type of conditions. Similarly, the production of chemical species by means of the plasma depends to a very great extent on the presence of pulsed current. For a DC voltage discharge, the quantity of charge associated with the pulsed current is of the same order of magnitude as the quantity of charge associated with the DC current. In DBD discharge, the quantity of charge associated with the synchronous current can remain well above the quantity of charge coming from the pulses, particularly under the low power conditions that are used for the method in question.

The effective impedance of the discharge giving the relationship between current and voltage can vary over time, and it depends strongly on the shape of the electrodes. The high voltage power supply is thus preferably regulated at DC in order to maintain the same discharge conditions and the same production of chemical species so as to ensure that the sterilizing effect is constant. For linear electrodes, the discharge conditions and the production of chemical species are a function of current per unit length.

In order to determine which conditions are in use, it is theoretically necessary to implement very high frequency acquisition over a large number of points in order to be able to distinguish the various components of the current. However, the inventors have shown that it is possible in practice to use only a few measurements that are more simple and therefore less expensive in order to measure the DC or synchronous current and the peak current associated with the various types of discharge current that have the major effect in the method of the invention, even when using low power conditions. This is particularly true for DBD discharge, even when the associated quantity of charge is small. Measuring peak current under such circumstances is thus particularly appropriate since it makes it possible to obtain a signal that is highly sensitive to the presence of pulses of this type, which is not possible merely by measuring mean current or power.

Under such circumstances, it is thus particularly useful to make use of peak current at least as the monitoring measurement, and better as the regulation parameter. Under such circumstances, it is necessary to provide an integration constant that is sufficiently large so as to avoid taking account of the “natural” fluctuations in the amplitudes of the pulses.

It is also possible to make use of an indirect measurement of current by measuring the charge on a capacitor. This provides a DC measurement even when using an AC power supply, since the capacitor discharges automatically. When the power supply is by means of a DC voltage, it is necessary to make provision for the capacitor to be discharged periodically.

Finally, a monitoring measurement can be performed by counting the number of pulse discharges that can be identified by current peaks of a few milliamps (mA) and of short duration (typically 100 ns) that occur over a given period of time, with the time density of such current peaks serving to determine the conditions in use. Naturally, the measurement system must be fast enough and accurate enough to be able to give the exact number of discharges.

The voltage signal applied to the high voltage electrode may be a DC signal or a squarewave signal of positive or negative sign, alternating, or even pulsed as a function of the discharge conditions selected for each of the stages. It is therefore preferable for the generator to have available a solution which provides the maximum amount of modularity in terms of amplitude and waveform.

By way of example, the generator may be implemented as shown in FIG. 3. A transformer 100 having a resonant frequency situated at low frequency, typically below 100 kilohertz (kHz) is powered by a low voltage DC source 102 that preferably operates as a current source via a transistor used as a controlled switch 104. The duration of the pulse 106 is adjusted so as to optimize break current, with the transformer serving both to raise voltage and as a filter, thereby delivering a sinusoidal individual pattern. The pulse repetition frequency is determined by the pulse generator 14 on the basis of a reference supplied by the control member 50. By repeating the pulse at the resonant frequency of the transformer, a signal 108 is obtained that is almost sinusoidal. By reducing the repetition frequency, pulse feed is obtained with an individual pattern that comprises a damped sinewave 110. To obtain a DC feed, it is necessary to place a rectifier system at the output from the transformer. It is also possible for all types of pattern, DC, sinewave, or damped sinewave, to introduce a latency time between the patterns corresponding to zero voltage at the output from the transformer in order to obtain a signal that has an envelope of rectangular type, e.g. of the type 112 for a sinewave pattern. The amplitude of the signal depends on the DC voltage applied to the transformer. The regulation can be performed using a current or a voltage measurement at 118 which is compared with a reference within a comparator 116 serving to act on the low voltage source 102. The inventors have demonstrated that this type of power supply of very simple design and low cost makes it possible to obtain a discharge that is stable. It also has the major advantage of making it possible to select between various types of high voltage signal merely by programming, while nevertheless remaining very simple. Within discharge conditions of a given type, until chemical saturation due to recombination of unstable species has been reached, increasing current serves to increase the production of species. A potential optimization of effectiveness for selected discharge conditions thus requires total current to be increased without changing conditions.

The simplest configuration for obtaining a corona discharge is a point-plane type configuration in which the set of electrodes is constituted by a point placed perpendicularly to a plane. This configuration makes it possible to determine a relationship between current and electric field that characterizes the various kinds of discharge conditions. By extending a point-plane configuration, it is possible to increase the number of points by placing a plurality of points on a common blade extending parallel to the plane. For discharge conditions defined on the basis of the point-plane configuration, it is thus possible to increase the total current at constant voltage without changing conditions, merely by increasing the number of points. This increase of current for constant voltage and discharge conditions is possible providing the points are electrically independent, i.e. providing their separation distance remains greater than 2d, where d is the interelectrode distance. At higher density, total current saturates for constant electric field.

Similarly, the inventors have been able to show that it is possible to increase the maximum total current under discharge conditions that remain unchanging by increasing the density of points above the electrical dependency threshold. For example, for DC voltage discharge, it is possible to push back the initiation of arcing which corresponds to a sharp change of conditions while increasing the density of points. Thus, it has been possible to obtain a current density under streamer conditions of about 162 microamps per centimeter (μA/cm) for an interpoint distance of 1 mm with an interelectrode distance of 10 mm, whereas the density is limited to 70 μA/cm when the distance between the points is 10 mm, as can be seen from Table 4 below.

TABLE 4
The effect of the number of points on maximum current prior to
reaching arcing conditions for an interelectrode distance of 10 mm

| Distance between points [mm] | Maximum current [μA/cm] |
|---|---|
| 10 | 70 |
| 5 | 100 |
| 2 | 125 |
| 1 | 162 |

It should be observed that changing over to arcing conditions gives rise to problems of stability, and increases problems of electromagnetic compatibility and mechanical strength for the electrodes, which makes arcing conditions more difficult to use.

Increasing point density thus serves to extend voltage ranges corresponding to each kind of discharge conditions. This increase in operating range corresponds to increasing the stability of each kind of conditions, thereby reducing constraints on regulation. The density limit is given by manufacturing constraints associated with the electrode and by the maximum voltage that can be delivered by the generator.

Tests performed by the inventors have shown that the higher the humidity in the vicinity of the article, the shorter is the length of time required for sterilization (see in particular Table 3 above). Furthermore, as shown by Table 5 below, an increase in the temperature of the surface to be sterilized also decreases the sterilizing effect.

TABLE 5
The effect of heating the sample
15 min treatment

| Temperature difference [° C.] | Living spores | |
|---|---|---|
| −5 | <10 | |
| 0 | <10 | |
| 5 | 10⁶ | (R) |

It is therefore necessary to maintain relative humidity as measured at the temperature of the article if this temperature differs from the temperature of the humidifying chamber, so as to keep it above a critical value which is estimated as lying around 50%. It is of interest to observe that there is no need to moisten articles or to give rise to uniform condensation: the article and the humidifier can remain at the same temperature. The inventors have also verified that sterilization is effective even on wet articles.

An electric discharge produces power which is dissipated in the gas in the discharge zone and in the electrodes. This heating can therefore raise the temperature of the surfaces to be sterilized, thus eliminating the sporicidal effect by reducing local relative humidity, as can be seen from Table 3 above. There is therefore a maximum level of power that should not be exceeded and that depends on the configuration, in particular for the second plasma discharge during the second stage Phb. Unfortunately, certain discharge conditions cannot be reached electrically without the instantaneous electrical power reaching some minimum value. This applies for example to DBD type discharge: there must necessarily exist some minimum power level being dissipated by the synchronous current before the high amplitude pulses associated with these conditions appear.

A first solution consists in reducing heating by using pulsed AC feed or feed having a rectangular envelope enabling the mean dissipated power to be reduced while conserving a sufficiently high level of instantaneous electrical power.

A second solution, when the humidifier is an evaporation humidifier, consists in maintaining the temperature of the humidifier at a temperature close to and slightly below the temperature at the surface to be sterilized in order to maintain constant relative humidity at the article. This configuration applies only to simple surfaces whose temperature can be measured directly or can be estimated on the basis of the temperature of the surrounding gas. In addition, it is necessary to make sure that there are no cold points between the humidifier and the discharge zone.

Another solution consists in using a vaporization humidifier to supersaturate the gas with water so that the relative humidity after heating remains sufficient. Under such circumstances, the discharge also serves to evaporate microdroplets created by the humidifier: vaporization is controlled so as to maintain the humidity at the temperature of the article at a level which is sufficient. This configuration requires accurate control of temperature gradients and operates only if the quantity of microdroplets needed does not exceed the limit that is acceptable for discharge.

The first stage Pha of bringing the reactor into equilibrium terminates when the sterilizing action of the species produced by the second plasma can actually begin, i.e. when chemical conditions both in terms of humidity and of chemical equilibrium more generally are favorable for sterilization. The supply of water vapor is provided by the flow of gas passing through the humidification chamber: it is therefore limited by the capacity of the humidification chamber to humidify at a given flow rate. The effect of discharging the first plasma is limited by the maximum rate of production in the discharge zone which is a function of flow rate. In general terms there therefore exists an optimum flow rate which needs to take account of two targets. In some cases, introducing humidity and discharging the first plasma need not take place simultaneously. At the end of stage Pha, the humidity measured at the sensor 46 serves to verify that the necessary humidity threshold has been reached.

The discharge of the first plasma is selected so as to minimize the length of time needed to bring the enclosure into equilibrium. The power must be selected in such a manner as to guarantee an acceptable temperature at the end of the treatment cycle. By way of example, this can imply applying power that decreases over time in order to make it possible to return to a lower temperature.

Discharge of the second plasma is selected as a function of its sporicidal action in the presence of humidity. Its power is limited to guarantee an acceptable temperature throughout its duration. Among various possibilities, the inventors have shown, for example (see Table 6 below), that the DC streamer conditions or the pulsed DBD discharge conditions provide sporicidal action that leads to decimal reduction times D measured on the spores of Bacillus subtilis that are of the order of a few minutes for powers of less than 1 watts per liter (W/L).

TABLE 6
Comparison between DC voltage discharge and DBD discharge
Living spores

| Time [min] | DC discharge | | DBD discharge | |
|---|---|---|---|---|
| 0 | 10⁶ | (R) | 10⁶ | (R) |
| 10 | 10³ | | 10⁵ | |
| 20 | <10 | | 10² | |
| 30 | <10 | | <10 | |

The sensor 46 serves to verify that the humidity is sufficient throughout the operation of stage Phb.

It should be observed that in the special case where the discharges of stages Pha and Phb can be produced simultaneously (different sources, superposable electrical signals, etc.), then it can be advantageous to cause stages Pha and Phb to overlap, with stage Phb beginning before the end of stage Pha. Thus, the filling of the treatment enclosure with the gas of stage Phb is already complete by the end of stage Pha, so stage Phb is then effective immediately.

Finally, the third stage Phc is a rinsing stage performed using a non-humidified gas. The main parameter is thus flow rate which depends on the volume of the enclosure.

The multiparameter sensor 46 placed at the outlet from the treatment enclosure 10 enables to determine when stage Phc has come to an end since that corresponds to the chemical composition of the gas returning to a composition that is acceptable for storage. The criterion is preferably based on measured levels of humidity and ozone as provided by this sensor. The ozone sensor is preferably a sensor that operates over an intermediate range, typically 1 ppm to 3000 ppm.

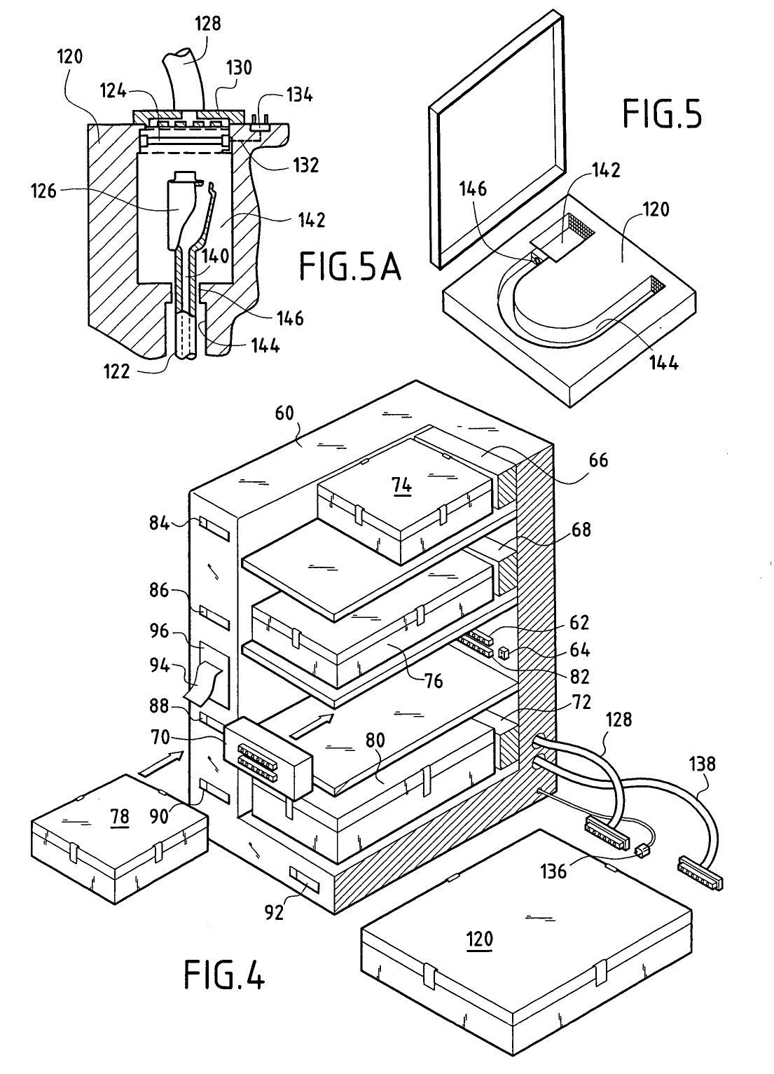

A first embodiment of a sterilization device implementing the above-described principles is shown in FIG. 4. It comprises a modular assembly having a central unit 60 with various types of treatment enclosure 74-80, 120 being fixed thereto. This modular configuration makes it possible to treat a set of enclosures that are matched in terms of number, shape, and volume to the articles which are to be treated either simultaneously or otherwise using a single central unit having one or more high voltage power supplies and a single gas management system (providing both supply and recovery) all of which is contained in the central unit. The use of a plurality of high voltage power supplies and a plurality of humidification chambers can enable the regulation system to be simplified and can allow different housings to be used asynchronously, and the use of a plurality of multiparameter sensors makes it possible to monitor the composition of the recovered gas prior to filtering in order to determine or to verify the time for associating with each stage. The sterilization zone may be of varying size or it may be standardized, depending on the user's requirements. By matching the shape and the volume of the zone to the articles which are to be sterilized, it is possible to optimize the flow of the sterilizing agent (its flow speed and its concentration) around the articles, and thus to ensure that treatment is uniform. The gas after treatment is then returned to said central unit via one or more gas exhaust inlets. This type of device can be made available since the low cost of implementing the method makes it possible to multiply the number of treatment zones, and thus to fragment the volumes under treatment.

This modular assembly comprises a common central unit containing the gas mixture source, one or more humidification chambers, and one or more high voltage power supplies. The central unit 60 has one or more gas outlets (e.g. 62) and a corresponding number of high voltage outputs (e.g. 64) feeding one or more treatment enclosures, each having its own plasma production zone 68, 70, 72 corresponding to the plasma production zone 10a and supplying sterilizing gas to a sterilization zone 76, 78, 80 corresponding to the sterilization zone 10b and containing the articles that are to be treated. The plasma production and sterilization zones may form two distinct zones of a common enclosure (e.g. the enclosures 74 and 130) or they may each be contained within a separate enclosure then referred to as the plasma production enclosure (as applies to enclosures 68, 70, 72) or the sterilization enclosure (as applies to the enclosures 76, 78, 80). Advantageously, all or part of the device as a whole is placed in a Faraday cage in order to limit the amount of interference that is created by the discharge.

Indicator and control means 84, 86, 88, 90 placed on the central unit 60 in register with the corresponding enclosures with which they are associated serve to inspect each enclosure individually, making sure that the sterilization cycle starts and the times of the various stages of treatment are adjusted while also adjusting the reference flow rate, and the regulation current in appropriate manner and also the waveform of the voltage signal, possibly also while also defining the composition of the gaseous mixture to be used. The volume of the enclosure determines the number and the sizes of the plasma sources used: it therefore has a direct influence on the reference flow rate and on the current. This reference also depends on the selected discharge conditions, and thus on the stage currently in progress.

The various commands are monitored by issuing a printed label 94 on a printer 96 integrated in the central unit 60. For each enclosure which carries a specific identity number, it is thus possible to mention on the label the date of treatment and the parameters of the sterilization cycle, in particular its duration. The enclosures may be provided with an automatic identification system, for example based on bar codes or on radiofrequency identification (RFID) type electronic labels 98a or on infrared communication (IRC) type labels, thus enabling the central unit to act via a corresponding reader 98b to determine automatically the values for the reference flow rate and for the regulation currents that are appropriate and to calculate the durations of the various stages of the sterilization cycle. The electronic labels can be placed inside the enclosure and most advantageously they can be provided with sensors enabling the sterilization cycle to be monitored, in particular chemical measurement sensors for the purpose of measuring humidity, ozone, pH, or nitrogen dioxide concentration, for example.

FIGS. 5 and 5A show an embodiment of a treatment enclosure that is more specifically adapted to sterilizing an endoscope and that is provided with a single plasma production zone.

This treatment enclosure 120 is characterized by a special shape for the electrodes constituting the plasma production zone, also serving to produce, in situ, chemical species that are sterilizing. With conventional sterilization techniques, the internal zones of articles to be sterilized can give rise to problems of sterilization if the active species have difficulty in reaching those zones. This problem is particularly acute for cavities or the insides of tubes, for example channels in endoscopes. The sterilization method of the invention is entirely suitable for being applied to sterilizing such cavities and it also makes it possible in simpler manner to resolve the problem of access to the internal zones of articles that are very elongate in shape.

The enclosure 120 is in the form of a box that can be sealed hermetically and whose inside space (the sterilization zone proper) is arranged as a function of the shape of the article 122 to be treated. Thus, in the example shown, the endoscope is curved flat inside the enclosure and a single plasma production zone 124 is defined in the vicinity of the head 126 of the endoscope. The vector gas is taken to the box of the central unit 60 via an external connection 128 and it is redistributed to the plasma production zone via a respective internal pipe 130. The electrodes in the plasma production zone are connected via a connection 132 to an external high voltage connector 134 in turn in connection with a corresponding compatible connector 136 of the common central unit 60. A connection 138 enables the gas to be exhausted towards said central unit 60 after treatment. In order to ensure that the inside surface of the endoscope channel 140 is properly sterilized, the sterilization zone comprises a first zone 142 surrounding the head of the endoscope 126 and maintained at a pressure that is slightly raised in order to guarantee a desired flow rate inside the channel 140, the remainder of the instrument being placed in a second zone 144 which is separated from the preceding zone by a passage 146 of determined diameter so as to allow the outside surface of the endoscope to be treated. By creating an annular constriction around the endoscope, this passage enables the first zone 142 to be maintained at a pressure that is slightly raised. In an alternative embodiment, it is possible to connect the end of the channel to a pump serving to establish a small amount of suction so as to cause flow to take place inside the channel and thus use the same plasma source.

Naturally, non-return check valves and/or antibacterial filters are provided at the interfaces with the box 100 so as to ensure that it remains sealed after being disconnected from the common central unit 60. Individual inspection of the box is provided by indicator and control means 92 placed on the central unit.

The improved method as described above is both simple in design since the enclosure has no need to withstand significant pressure differences and the gas feed system is simplified, and simple to use there are no chemicals to be handled before and after the sterilization cycle and the risk of pollution is small. In addition, the high voltage and low frequency power supply system is simple in structure and can be adapted to various configurations.

The applications proposed relate essentially to the medical field, but the method can naturally be extended to many other industrial applications, for example to the pharmaceutical or food industry fields.

1. A method of sterilizing at least one article (20) by means of a plasma and in the presence of humidity (14) using a non-biocidal gas containing oxygen and nitrogen, the article being placed outside the discharge in a sealed treatment enclosure (10) that is subjected substantially to atmospheric pressure, the method being characterized in that it comprises the following steps:
introducing the humidified non-biocidal gas into the treatment enclosure;
creating a first plasma discharge A for a determined duration enabling the effectiveness of the sterilizing species created during the following stage to be guaranteed within the entire enclosure;
creating a second plasma discharge B during a determined duration enabling said article to be sterilized; and
rinsing the treatment enclosure during a determined duration so as to guarantee that it contains a non-polluting atmosphere when the enclosure is subsequently opened.

2. A sterilization method according to claim 1, characterized in that said determined durations are also calculated as a function of the volume of the treatment enclosure.

3. A sterilization method according to claim 1, characterized in that the end of the rinsing step is detected by crossing a threshold as measured by a multiparameter sensor (46) placed at the outlet from the treatment enclosure.

4. A sterilization method according to claim 1, characterized in that the first plasma discharge and the introduction of humidity take place simultaneously.

5. A sterilization method according to claim 1, characterized in that the first and second plasma discharges make use of the same plasma source (12).

6. A sterilization method according to claim 5, characterized in that the first and second plasma discharges are of different kinds so as to enable each of the stages to be optimized separately.

7. A sterilization method according to claim 1, characterized in that the flow rate of the non-biocidal gas is different in the various stages.

8. A sterilization method according to claim 1, characterized in that the discharge conditions for the first and second plasmas are selected by the type of pattern of the voltage signal (sinewave, damped sinewave, or DC), the repetition frequency of the pattern, and the total reference current.

9. A sterilization method according to claim 1, characterized in that the discharge conditions used are controlled by detecting peak current.

10. A sterilization method according to claim 9, characterized in that said detection is performed with a passband width of the same order as the frequency between pulses.

11. A sterilization method according to claim 1, characterized in that the repetition frequency of the pattern for a latency time between patterns is used for limiting temperature rise at the article for sterilizing while conserving the same discharge conditions.

12. A sterilization method according to claim 1, characterized in that provision is also made for an evacuation humidifier to be temperature-stabilized at a temperature that is slightly below the temperature of the article in order to compensate for the temperature rise due to the discharge.

13. A sterilization method according to claim 1, characterized in that provision is also made to control the effectiveness of a vaporizer so as to compensate for the temperature rise due to the discharge.

14. A sterilization method according to claim 1, characterized in that the high voltage power supply is taken from a pulsed low voltage power supply (102) feeding a transformer (100) used as a filter and as voltage step-up means.

15. A sterilization method according to claim 14, characterized in that controlling the repetition rate of the low frequency pulses serves to define the individual pattern and to introduce a latency time that is adjustable.

16. A sterilization method according to claim 14, characterized in that the low voltage pulse repetition frequency is less than the resonant frequency of the transformer.

17. A sterilization method according to claim 14, characterized in that the low voltage pulse repetition frequency is equal to the resonant frequency of the transformer.

18. A sterilization method according to claim 14, characterized in that the power supply is regulated on the basis of a current measurement, preferably a DC measurement for a DC power supply, or a synchronous measurement for an AC power supply.

19. A sterilization method according to claim 18, characterized in that said current measurement is performed via a resistance or by measuring the charge on a capacitor.

20. A sterilization method according to claim 19, characterized in that, for a DC power supply, the capacitor is discharged by being connected periodically to ground.

21. A sterilization method according to claim 18, characterized in that the power supply is regulated on the basis of measuring a peak current.

22. A sterilization method according to claim 21, characterized in that the signal used for regulation is smooth with a time constant longer than 100 ms, and preferably of 1 s.

23. A sterilization method according to claim 1, characterized in that the electrodes are made from a blade having one or more points parallel to a plane or cylindrical surface that acts as a backing electrode.

24. A sterilization method according to claim 23, characterized in that the number of points is selected in such a manner as to facilitate the use of the desired different discharge conditions during treatment.

25. A sterilization method according to claim 1, characterized in that the first and second plasma discharges overlap in such a manner that creation of the second plasma B begins before creation of the first plasma A terminates.

26. A sterilization device comprising a plurality of treatment enclosures, each treatment enclosure having at least one plasma production zone connected in optionally fixed manner to at least one sterilization zone, the plasma production zones being connected to a common central unit containing at least the first source of non-biocidal gas, the humidification chamber, the system for recovering gas residues, and the high voltage power supply, which device is characterized in that the central unit has as many high voltage power supplies as there are outlets enabling enclosures to be treated simultaneously with different discharge conditions being applied thereto.

27. A sterilization device according to claim 26, characterized in that the sterilization zone is pressurized to a small extent so as to enable flow to take place in fine capillaries.

28. A sterilization device according to claim 26, characterized in that the common central unit includes a multiparameter sensor for each connection part enabling the gas composition leaving an enclosure to be monitored prior to filtering.

29. A sterilization device according to claim 28, characterized in that said sensor enables humidity and ozone concentration to be measured.
